(19) United States
(12) Patent Application Publication
(10) Pub. No.: US 2022/0088331 A1
(43) Pub. Date: Mar. 24, 2022

(54) ANALYSIS UNIT FOR DETECTING A DISCONNECTION AT A VENTILATOR

(71) Applicant: Drägerwerk AG & Co. KGaA, Lübeck (DE)

(72) Inventors: Lorenz KAHL, Lübeck (DE); Przemyslaw GDANIEC, Lübeck (DE)

(21) Appl. No.: 17/479,378

(22) Filed: Sep. 20, 2021

(30) Foreign Application Priority Data
Sep. 22, 2020 (DE) 10 2020 124 585.8

(51) Int. Cl. A61M 16/00

(52) U.S. Cl. CPC A61M 16/024; A61M 16/0003; A61M 2205/14; A61M 2016/0027; A61M 2230/42; A61M 2016/0039

(57) ABSTRACT

A ventilator analysis unit (100) detects a disconnection of a pneumatic connection to a ventilated patient. A data acquisition module (110) receives gas flow data (112) indicating gas flow at the transition to the patient and gas pressure data (114) indicating a transition gas pressure. The memory module (120) provides a disconnection value function (122) describing the transition gas flow transition gas pressure association with a disconnection value (132). The calculation module (130) calculates a disconnection value based on the gas flow data and the gas pressure data via the current disconnection value function and determines a disconnection number (134) from a chronological sequence of correspondingly calculated, current disconnection values. The calculation module further indicates a presence of a disconnection of the pneumatic connection via a corresponding output (140) if the disconnection number reaches a predefined threshold value or is above the predefined threshold value over a predefined time period.

### CROSS REFERENCE TO RELATED APPLICATIONS

This application claims the benefit of priority under 35 U.S.C. § 119 of German Application 10 2020 124 585.8, filed Sep. 22, 2020, the entire contents of which are incorporated herein by reference.

### TECHNICAL FIELD

The present invention pertains to an analysis unit for a ventilator for detecting a disconnection of a pneumatic connection between the ventilator and a patient to be ventilated by the ventilator. The present invention pertains, furthermore, to a ventilator with such an analysis unit. Finally, the present invention also pertains to a process for detecting a disconnection of a pneumatic connection between a ventilator and a patient to be ventilated by the ventilator and to a computer program with a program code for carrying out such a process.

### TECHNICAL BACKGROUND

It is known that the connection between a ventilator and a patient to be ventilated by the ventilator can be checked in an automated manner. Damage to the patient due to an improper connection, for example, due to a disconnection of the patient from the ventilator, shall be avoided hereby.

Ventilation times over several days or weeks are common especially in the case of ventilation in an intensive care unit. The ventilation cannot always be monitored by the health care staff directly at the ventilator and/or at the patient, so that an error-free, automated alarm generation is especially important there.

It is described in U.S. Pat. No. 8,322,339 B2 how an error in the connection between a patient and a ventilator is detected in an automated manner by checking a pressure signal and by the subsequent checking of a flow signal, taking into consideration correspondingly predefined threshold values.

### SUMMARY

An object of the present invention is to provide an improved analysis for detecting such a disconnection, particularly an especially reliable detection and/or an especially rapid detection of such a disconnection.

To accomplish this object, an analysis unit is provided according to the present invention for a ventilator for detecting a disconnection of a pneumatic connection between the ventilator and a patient being ventilated by the ventilator, with a data acquisition module, with a memory module and with a calculation module.

The data acquisition module is configured to receive gas flow data and gas pressure data, wherein the gas flow data indicate a gas flow present at the transition to the patient through the ventilator and wherein the gas pressure data indicate a gas pressure present at the transition to the patient through the ventilator.

The memory module is configured to provide a current disconnection value function, which describes an association between the gas flow present at a transition and the gas pressure present at the transition with a disconnection value to be associated (to be assigned).

The calculation module is configured to receive the gas flow data, the gas pressure data and the current disconnection value function and to calculate a current disconnection value based on the gas flow data and the gas pressure data via the current disconnection value function, especially to calculate a single current disconnection value, wherein the calculation module is further configured to determine a disconnection number from a chronological sequence of correspondingly calculated current disconnection values, especially to determine a disconnection number at recurring time intervals. The calculation module is further configured to indicate the presence of a disconnection of the pneumatic connection via a corresponding output if the disconnection number reaches a predefined threshold value or it is above this predefined threshold value over a predefined time period.

It was found within the framework of the present invention that the taking into consideration of different typical ventilation situations makes it necessary to take into consideration the gas flow and the gas pressure to detect a disconnection. It was found, in particular, that the disconnection value function must be dependent on these two variables in order to detect possible reasons, for example, for a high gas flow or for a low gas pressure and in order to thereby avoid a false disconnection alarm.

False alarms are advantageously avoided by the analysis unit according to the present invention during the automated disconnection detection, so that real problems in the connection between the patient and the ventilator can be detected especially quickly and reliably. In particular, the avoidance of false alarms ensures that justified alarms are pursued especially attentively, for example, by the health care staff.

Furthermore, changes in the gas pressure and/or gas flow within the framework of, for example, ventilation maneuvers or of a changed ventilation situation are taken into consideration especially advantageously due to the use of the disconnection number dependent on the gas flow data and gas pressure data, without triggering a false alarm thereby. In particular, the disconnection value function can have been determined for the concrete ventilator employing the disconnection value function, so that special characteristics of this ventilator, of this type of ventilator or of the hose system, e.g., the type of humidifier, the type of heater, the filter type, patient interface or the like, are taken into consideration within the disconnection value function.

That the disconnection value function is dependent on the gas flow data and on the gas pressure data means that a change in the gas flow can lead to a change in the disconnection value function if the value of the gas pressure is maintained at a constant value and a change in the gas pressure can also lead to a change in the disconnection value function if the value of the gas flow is maintained at a constant value. The disconnection value function may not be continuous in this case at least at one point or along a section, and it may have, in particular, a jump.

The disconnection value function is preferably selected to be such that a change in the gas flow while the gas pressure is maintained at a constant value can lead to at least three different values for the disconnection value function. As an alternative or in addition, the disconnection value function is preferably selected to be such that a change in the gas pressure while the gas flow is maintained at a constant value can lead to at least three different values for the disconnection value function. It is advantageously ensured in these two examples for the disconnection value function that the disconnection value function describes not only a threshold value for the gas flow and for the gas pressure via a corresponding step function. A physiologically motivated dependence of the disconnection probability on corresponding gas flow data and gas pressure data and the correlation thereof is thus especially advantageously described according to the present invention.

The gradient of the disconnection value function is preferably not constant along a straight line with constant gas flow or with constant gas pressure within a gas flow-gas pressure diagram.

Due to the disconnection value function depending on the gas flow data and on the gas pressure data, only a single current disconnection value, in which the gas flow data and the gas pressure data are taken into consideration, is determined.

The disconnection value function may also depend on additional data, in addition to depending on the gas flow data and on the gas pressure data. In an embodiment according to the present invention, the disconnection value function depends, furthermore, on a measured temperature, a measured humidity and/or on a gas mixture.

The determination of the disconnection number from a plurality of current disconnection values calculated over a chronological sequence makes it advantageously possible to take into consideration a chronological development of gas flow and gas pressure and it makes it possible as a result not to trigger a disconnection alarm already on the basis of a short-term measurement error or based on a short-term irregularity of the ventilation, for example, in case of a cough or hiccup of the patient.

A breathing cycle preferably represents an at least nearly closed circuit in a gas flow-gas pressure diagram. The disconnection number of such a breathing cycle consequently arises from the disconnection value calculated for corresponding points of this circuit. The disconnection value function is selected to be such that the disconnection number rises when no circuit that is at least nearly closed is present for a breathing cycle, but a current state has moved markedly away from previously assumed states in the gas flow-gas pressure diagram. The concrete structure of the disconnection value function is formed on the basis of physiological relationships and will be explained in detail within the framework of FIG. 2 in the description of the figures.

The gas flow data and gas pressure data received according to the present invention are gas flow data and gas pressure data taken into account in some embodiments according to the present invention, especially gas flow data and gas pressure data taken into account on the basis of a hose system that is currently present for the ventilation. In further alternative or additional embodiments according to the present invention, the gas flow data and gas pressure data are not data determined at least partially via a sensor in the hose system, but data measured internally, especially in the ventilator, which are taken into account via a model, for example, via a hose system-dependent model, to obtain the received gas flow data and/or gas pressure data. Finally, data measured at least partially in the ventilator may also be used according to the present invention without further taking into account as gas flow data and/or as gas pressure data if these data contain information concerning the gas flow and/or the gas pressure at the transition between the patient and the ventilator and they indicate in this sense the gas pressure and/or the gas flow in this area.

The modules of the analysis unit according to the present invention may be arranged adjacent to one another, especially in a common housing. As an alternative, these modules may be arranged at least partially at spaced locations from one another, for example, they may be configured by different processors. The modules of the analysis unit according to the present invention are preferably configured by a common processor. They are separated from one another at least at the software level.

The gas pressure is preferably the gas pressure in the area of the airways of the patient and hence essentially the so-called airway pressure or Paw. As an alternative or in addition, the taking into account of the airway pressure can be calculated with a property of the ventilation system, e.g., of the hose, in order to determine the gas pressure that is to be subjected to further processing. For example, a maximum possible pressure drop in the exhalation hose area can thus be taken into consideration in order to avoid excessively high measured values for the gas pressure, for example, on the basis of system errors. The gas flow is preferably the gas flow in the area of the airways of the patient and is therefore essentially the so-called patient flow.

Preferred embodiments of the analysis unit according to the present invention will be described below.

The data acquisition module is configured in an especially preferred embodiment to receive the gas flow data and gas pressure data essentially in real time, and the calculation module is configured to calculate the current disconnection value essentially in real time. A disconnection can be detected especially rapidly in an automated manner in this embodiment, and, in particular, the disconnection number can be determined on the basis of especially current values for the disconnection value. The time elapsing between the receipt of the data by the data acquisition module and the calculation of the disconnection value is preferably shorter than 2 sec, especially shorter than 1 sec, and especially preferably shorter than 0.1 sec.

In an especially advantageous variant of the above embodiment, the calculation module is configured to calculate the respective current disconnection values over a plurality of breaths of the patient and to determine the disconnection number over this plurality of breaths. The disconnection number is preferably determined in this case essentially in real time anew for newly calculated disconnection values. In particular, the disconnection number is preferably also determined for disconnection values that were already used at least partially for the determination of the preceding disconnection number.

The disconnection value function especially preferably depends on an existing ventilation mode of the ventilator, on an existing patient category of the patient, on a ventilation device being used and/or on existing ventilation parameters, especially the existing positive end-expiratory pressure (PEEP) and/or the inspiratory pressure set at the ventilator. The disconnection value function currently used for the calculation of the disconnection value is likewise changed in case of a change of the ventilation mode. It can thus be detected especially reliably whether a current value pair of gas pressure and gas flow indicates a disconnection within this ventilation mode or not. The ventilation mode may indicate, for example, whether a pressure- or volume-controlled ventilation is currently being carried out. The patient category can describe, for example, whether a newborn, a child or an adult is currently being ventilated. The ventilation device used may describe, for example, a hose system being used, a hose size, a patient connection, for example, via prongs, or the like.

In a variant of the above embodiment, the ventilation mode can indicate that an air shower shall be reliably avoided, for example, based on hygienic guidelines, in the presence of a disconnection. A maximum gas flow of the ventilator, which cannot be exceeded, is predefined for this ventilation mode. A disconnection value function associated with this ventilation mode preferably changes after reaching the maximum gas flow at time intervals such that a sustained status at the maximum gas flow leads to an accelerated increase in the disconnection number due to increasing disconnection value. It is advantageously ensured hereby that there will not be a considerable delay in the detection according to the present invention of the disconnection due to a maximum gas flow set at a low value. Furthermore, it is advantageously avoided in this variant that a disconnection is erroneously detected immediately on the basis of the high gas flow due to sneezing of the patient or the like.

The calculation module is configured in another embodiment to determine a reading mode from a predefined plurality of reading modes and to use it for the reading of the gas flow data and of the gas pressure data. The determination of the reading mode is preferably carried out on the basis of a received input, which indicates a ventilation mode of the ventilator. A preset reading mode is, for example, the use of a gas flow measured last and of a gas pressure measured last in order to calculate the disconnection value on the basis of these two values. In a first alternative reading mode, maximum gas flows and/or maximum gas pressures, especially measured maximum gas flows and/or gas pressures measured in a predefined past time interval, of a periodic gas flow curve and/or gas pressure curve are used in order to calculate with these the disconnection value. This first alternative reading mode is especially advantageous for a high-frequency ventilation, as it is known, for example, in the case of newborns, during which the measured gas pressure and the measured gas flow are not in phase. A typical ventilation frequency for such a high-frequency ventilation is between 5 Hz and 15 Hz. In a second alternative reading mode, a distance between two points of a gas flow curve (time course) and/or of a gas pressure curve (time course), which distance recurs over time, is used to find the same distance within the corresponding other curve and thereby to carry out a comparison of the curves. Based on the comparison of the curves, individual measurement points or systematically recurring measurement points, which are incorrect, can be corrected, so that correspondingly corrected values for the gas pressure and for the gas flow are used to determine the disconnection value. This is especially advantageous if a sensor is operated in the range of its saturation, so that areas of the gas pressure curve and/or of the gas flow curve are cut off.

A change between two reading modes from the plurality of reading modes can be triggered in an automated manner and/or via a user input in an especially preferred variant of the above embodiment.

In an advantageous embodiment, the disconnection value function is based at least partially on a gas flow that is the maximum gas flow to be currently expected, and which depends on the currently present gas pressure. The disconnection value function depends in this embodiment at least partially on a pressure-dependent value for the maximum gas flow to be expected, which is compared with the currently occurring gas flow by the disconnection value function.

In another advantageous embodiment, the disconnection value function is based at least partially on a minimum gas pressure to be expected. A drop in the present gas pressure indicated by the gas pressure data to below the minimum gas pressure to be expected leads to a disconnection value, which is a dominant component in the taking into account with other disconnection values for the determination of the disconnection number and indicates the presence of a disconnection already after the determination of a few such disconnection values and thereby leads to the corresponding output. Fewer than five calculated disconnection values, especially preferably two calculated disconnection values, with a respective gas pressure below the gas pressure to be expected, are preferably sufficient to lead to a disconnection number that indicates the presence of a disconnection due to the fact that the predefined threshold value is exceeded.

In an especially advantageous embodiment, the analysis unit further has an input module, which is configured to receive a user input via an input interface and to provide a number of input data indicated by the user input. The user can advantageously influence in this embodiment the disconnection value function used for the automated detection of the disconnection. Thus, the user input may pertain to a selection of a disconnection value function from a predefined group of possible disconnection value functions. As an alternative or in addition, the user input may pertain to a sensitivity, which indicates how rapidly corresponding values for the gas flow data and/or gas pressure data lead to the output that a disconnection is present. A higher sensitivity accordingly also increases the risk of a false alarm and hence the risk of an unnecessary noise generation during the ventilation of a patient.

In a preferred variant of the present embodiment, the user input pertains to a manual analysis of the predefined threshold value for the disconnection number. For example, the sensitivity of the output can be set by setting the predefined threshold value. A high sensitivity can thus lead to a low threshold value and hence to a rapid output, which also indicates, however, a false alarm with a higher probability than in the case of a high threshold value for a low sensitivity. The manual analysis of an output pertaining to the presence of a disconnection is preferably used by the analysis unit in order to adapt the disconnection value function and/or the predefined threshold value. An incorrect indication of the presence of a disconnection can thus lead to an increase in the predefined threshold value in order to avoid such a false alarm in the future. As an alternative or in addition, the curve of the gradients of the disconnection value function can be adapted such that the disconnection values calculated previously for the adapted disconnection value function would not have led to a corresponding input.

In an especially preferred embodiment, the analysis unit has, furthermore, a modeling module, which is configured to provide the disconnection value function as a function of past gas flow data, of past gas pressure data and/or of provided input data and to make it available to the memory module, wherein the memory module is further configured to replace the past current disconnection value function by the adapted disconnection value function. The modeling module makes possible in this embodiment an especially reliable determination of whether a disconnection is present on the basis of the disconnection number, because the underlying disconnection value function is adapted by a data history and is especially reliable as a result. In particular, the disconnection value function is preferably adapted by a treatment history for the treatment of the corresponding patient. The disconnection value function can advantageously be adapted thereby to the patient-specific breathing cycle. The disconnection value function is preferably adapted by the data history at regular time intervals, especially continuously.

For example, an automated checking, which checks whether a state was present in the past that indicates with certainty that a disconnection is present with certainty or that a connection is present with certainty, can be carried out for the automatic adaptation of the disconnection value function. It is possible hereby to check decisions made in the past based on the disconnection value function on whether a disconnection was present in order to learn from these. A connection is indicated with certainty if a subsequent exhalation was detected after a plurality of breaths. A connection may also be indicated by the fact that a minimum pressure is exceeded. A connection can be indicated especially advantageously by a corresponding user input.

Based on such an analysis of past decisions that a connection was present, a reliable association of data sets is possible between “connected” and “disconnected;” in particular, a decision that a connected state was present can be verified retrospectively with certainty. Such an association of past data pairs of gas flow data and gas pressure data may form a basis for the calculation of the disconnection value function. This function of gas flow and gas pressure can thus be dependent on a quotient of the probability distribution for the presence of this data pair in the connected state, and of the probability distribution for the presence of this data pair in the disconnected state. In particular, this function can be formed by the logarithm of this patient. The probability distribution for the data pair in the disconnected state can be for this a probability distribution predefined prior to the operation of the ventilator or a probability distribution predefined on the basis of a calibration. The probability distribution for the data pair in the connected state may be a predefined probability distribution, which is preferably adapted on the basis of past measured data pairs during a connected state.

User inputs are preferably analyzed within the framework of a calibration of the analysis unit according to the present invention in order to make an association with corresponding test data with certainty that a connection is currently present or that a disconnection is currently present. Such a calibration can be used to generate a predefined disconnection value function or to adapt the modeling module to a ventilator and/or to a hose system and/or to a patient.

A type of adaptation of the disconnection value function can preferably be set by means of a user input. For example, the user can select whether a rapid adaptation, for example, an adaptation after at most 10 minutes, or a slow adaptation, for example, an adaptation after at least 2 hours, is desirable. Finally, the user can activate or deactivate the automated adaptation of the disconnection value function manually via a user input in one example.

The past time period that shall be used for the adaptation of the disconnection value function can be set by means of a user input in another exemplary embodiment. For example, it can be set whether the last 2 hours, the last 10 hours or the last 24 hours of the ventilation will be analyzed for the adaptation. An analysis unit preferably indicates the time period currently being used for the adaptation.

An intensity of the adaptation can preferably be set via a user input, in which case the user input indicates the change in the adaptation that is to be made as a percentage. The user input may indicate, for example, discrete steps of the adaptation intensity, e.g., a slight, moderate or major adaptation.

In an especially advantageous variant of the preceding embodiment, the adaptation of the disconnection value function by the modeling module is based on a processing of the disconnection value function, especially of the current disconnection value function, by a neuronal network. The neuronal network receives in this case the past gas flow data, the past gas pressure data and/or provided input data in order to provide the adapted disconnection value function based on these. An internal checking of whether the adapted disconnection value function has worsened the past disconnection value function and/or whether a false alarm was triggered by the adapted disconnection value function and a corresponding further adaptation of the disconnection value function is therefore necessary can always be carried out in this case via a user input.

In the area of the neuronal networks, the determination of the disconnection value function is a classification problem. The presence of one of two possible classes is inferred in this case on the basis of input data, which are the gas flow data and the gas pressure data. Numerous implementations of such classification problems are known to the person skilled in the art in the area of the neuronal networks. These neuronal networks are based in this case basically on activation functions at a particular nodal point of the network, which have at least one initially unknown term, e.g., an unknown scaling and/or an unknown summand. The learning process of the network is based on the fact that such terms, i.e., for example, scalings and/or summands, are adapted in the course of time via so-called cost functions of the network, and thus they lead to an adapted output value, namely, in this case to an adapted disconnection value function.

Furthermore, a minimal disconnection value function is stored for the modeling module in an especially preferred example of the preceding embodiment and/or of the preceding variant of this embodiment, wherein the modeling module is configured to provide the minimal disconnection value function as the adapted disconnection value function if a predefined function characteristic of the disconnection value function adapted by the modeling module exceeds a predefined characteristic threshold value. It is advantageously ensured in this embodiment that the adapted current disconnection value function does not differ so very much from the originally set disconnection value function and a safety risk could consequently arise for the patient based on an automatic adaptation. The minimal disconnection value function therefore represents a safety function, which meets clinical requirements imposed on the automatic detection of a disconnection, so that it is ensured that the patient's safety is not compromised by the automatic adaptation of the disconnection value function being used. The minimal disconnection value function is preferably supplemented by a stored maximal disconnection value function, which is provided as an adapted disconnection value function, if an additional predefined characteristic threshold value is exceeded. Such a maximum disconnection value function ensures that the adapted disconnection value function used by the analysis unit always remains in a range that meets the clinical requirements imposed on the automatic detection of a disconnection. It is thus ensured in an especially reliable manner that the patient's safety is not compromised by the automatic adaptation of the disconnection value function used. The predefined characteristic threshold value may pertain, for example, to a gradient threshold value, a pressure threshold value, a flow threshold value, a pressure difference threshold value and/or a flow difference threshold value for the adapted disconnection value function.

The disconnection value function used at the start-up of the analysis unit and/or the adaptation of the disconnection value function within the operation is preferably based on calibration data determined during a calibration of the analysis unit. The use of such calibration data leads to an especially reliable detection of the disconnection by the analysis unit. In particular, the adaptation of the disconnection value function can take place especially reliably on the basis of physiological data.

According to a second aspect of the present invention, a ventilator with an analysis unit according to at least one of the above embodiments is proposed for accomplishing the above-mentioned object.

The ventilator according to the present invention comprises the analysis unit according to the first aspect of the present invention and consequently all the advantages of this analysis unit.

In an especially advantageous embodiment, the ventilator has, furthermore, a sensor device, which is configured to detect and to provide the gas flow data and the gas pressure data. The configuration of the sensor device and hence a course of the gas flow data and of the gas pressure data, which course is expected as a result, can be taken into consideration in this embodiment in the determination of the disconnection value function in an especially advantageous manner.

In an alternative or additional embodiment, the ventilator is configured to take into account the gas flow data and the gas pressure data in a suitable manner on the basis of the known sensor device in order to provide data taken into account correspondingly for the data acquisition module according to the present invention. The taking into account may comprise, for example, a conversion of the data detected by the sensor device into the data probably occurring within a hose device of the ventilator. In particular, the variables measured by the sensor device can be taken into account via stored parameters to be taken into account in order to arrive thereby at the provided gas flow data and gas pressure data.

In another alternative or additional embodiment, the provided gas flow data and/or the provided gas pressure data are determined from at least one other variable describing the gas dynamics within the hose device. Thus, heating of an area of the hose system, the concentration of a breathing gas within the hose system, a pressure difference at a diaphragm, a speed of a blower within the ventilator or the like may be used to determine the gas flow data and/or to determine the gas pressure data.

According to a third aspect of the present invention, a process for detecting a disconnection of a pneumatic connection between a ventilator and a patient to be ventilated by the ventilator is proposed to accomplish the above-mentioned object. The process according to the present invention has the following steps here:
receipt of flow data and gas pressure data, wherein the gas flow data indicate a gas flow present at the transition to the patient through the ventilator and wherein the gas pressure data indicate a gas pressure present at the transition to the patient through the ventilator;
provision of a current disconnection value function, which describes an association between the gas flow present at the transition and the gas pressure present at the transition with a disconnection value to be assigned;
calculation of a current disconnection value based on the gas flow data and the gas pressure data via the current disconnection value function;
determination of a disconnection number from a chronological sequence of correspondingly calculated, current disconnection values; and
indication of the presence of a disconnection of the pneumatic connection if the disconnection number reaches a predefined threshold value or is above this predefined threshold value over a predefined time period.

The process according to the present invention is carried out by the analysis unit according to the first aspect of the present invention and thus it has the same advantages. In particular, the process according to the present invention makes possible an especially reliable determination of a disconnection between a patient and a ventilator due to the use of a disconnection value function, which depends on the current gas flow and on the current gas pressure.

The process according to the present invention is preferably carried out continuously, i.e., at times immediately following one another. The receipt of the gas flow data and gas pressure data may also take place when the disconnection number has not yet been determined for the preceding course of the process.

All steps of the process according to the present invention are carried out preferably essentially in real time. A time of preferably less than 10 sec, especially less than 5 sec, and especially preferably less than 2 sec elapses between the first process step, i.e., the receipt of the gas flow data and gas pressure data, and the final process step.

In an advantageous embodiment, the determination of the disconnection number does not take place after each calculation of the current disconnection value, but only after a predefined number of calculated current disconnection values. The predefined number preferably comprises fewer than 20 disconnection values, especially fewer than 10 disconnection values, and especially preferably fewer than 6 disconnection values.

According to a fourth aspect of the present invention, a computer program with a program code for carrying out a process according to the third aspect of the present invention is proposed for accomplishing the above-mentioned object. The program code is executed on a computer, on a processor or on a programmable hardware component. A plurality of steps of the process according to the present invention are carried out preferably by a shared computer, by a shared processor or by a shared programmable hardware component. The individual steps are preferably separated in this case at least at the software level by corresponding software blocks. All steps of the process according to the present invention are carried out especially preferably on a shared computer, on a shared processor or on a shared programmable hardware component.

The present invention shall now be explained in more detail on the basis of advantageous exemplary embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of an analysis unit according to a first aspect of the present invention;

FIG. 2 is a diagram of a first exemplary embodiment of a disconnection value function for the analysis unit according to the first aspect of the present invention;

FIGS. 3a is a diagram of a second exemplary embodiment of a disconnection value function;

FIGS. 3b is a time curve (time course) of the disconnection number for the analysis unit according to the first aspect of the present invention;

FIG. 4 is a schematic view of a second exemplary embodiment of the analysis unit according to the first aspect of the present invention; and

FIG. 5 is a flow chart of an exemplary embodiment of a process according to a third aspect of the present invention.

### DESCRIPTION OF PREFERRED EMBODIMENTS

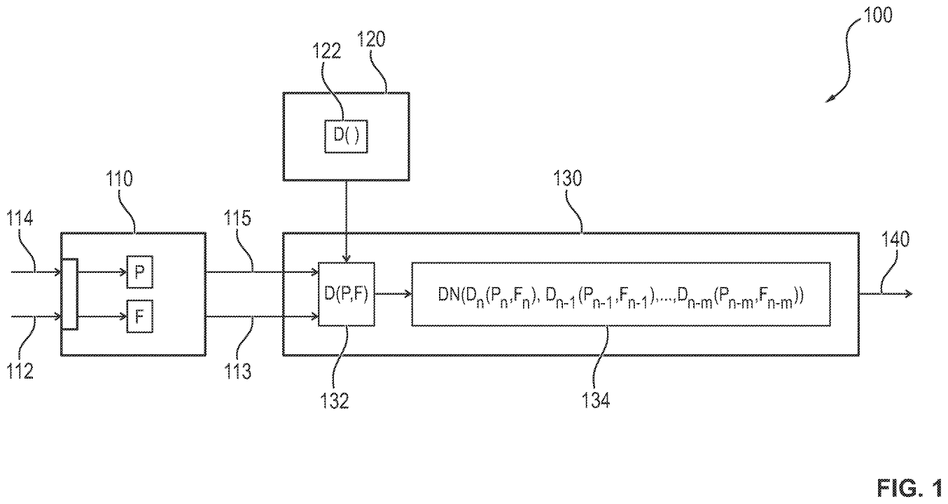

Referring to the drawings, FIG. 1 shows a schematic view of a first exemplary embodiment of an analysis unit 100 according to a first aspect of the present invention.

The analysis unit 100 is configured for a ventilator for detecting a disconnection of a pneumatic connection between the ventilator and a patient to be ventilated by the ventilator. The analysis unit 100 has for this a data acquisition module 110, a memory module 120 and a calculation module 130.

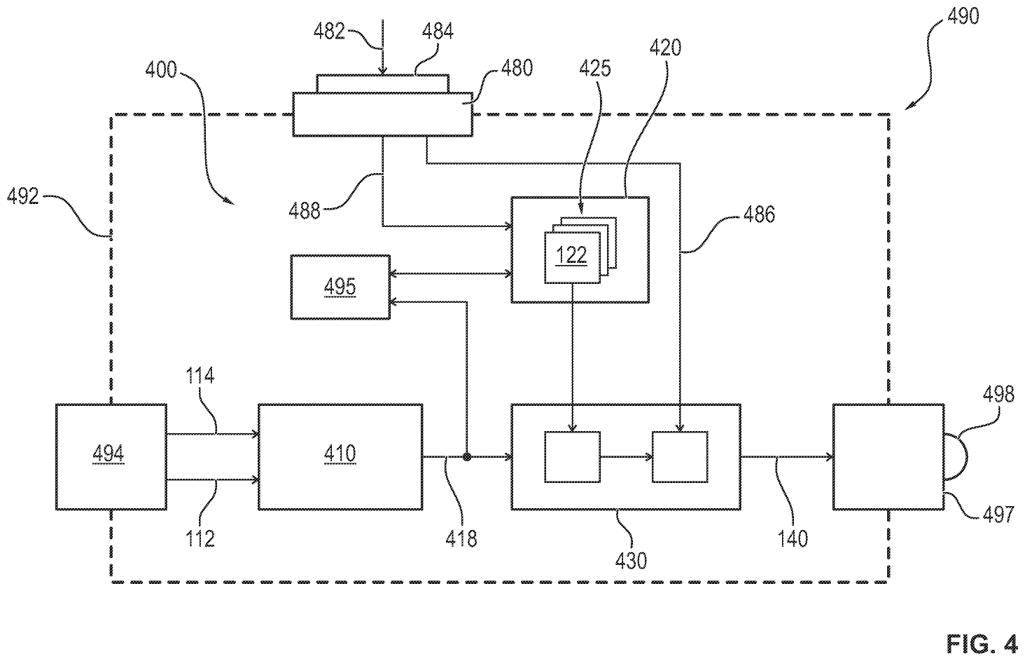

The data acquisition module 110 is configured to receive gas flow data 112 and gas pressure data 114, wherein the gas flow data 112 indicate a gas flow present at a transition to the patient through the ventilator and wherein the gas pressure data 114 indicate a gas pressure present at the transition to the patient through the ventilator. These data 112, 114 are received according to the present invention by the data acquisition module and are processed further such that they can be read or received by the calculation module 130. The data acquisition module 110 filters the received data such that a sequence of currently present gas pressures P and of currently occurring gas flows F are read from the received data 112, 114 in order to forward them to the calculation module via a gas pressure signal 115 and via a gas flow signal 113. The processed currently occurring values for the gas pressure and for the gas flow are outputted, especially outputted continuously, in an exemplary embodiment shown in FIG. 4 within a signal. The communication between the data acquisition module 110 and the calculation module 130 is a cable-based communication in the exemplary embodiment shown. This communication takes place in a wireless manner (wirelessly) in one exemplary embodiment, not shown.

The memory module 120 is configured to provide a current disconnection value function D 122, which describes an association between the gas flow F present at the transition and the gas pressure P present at the transition, on the one hand, and a disconnection value D(P, F) 132, on the other hand. The memory module 120 is connected to the calculation module 130 in order to make it possible to output the disconnection value function directly to the calculation module. In one exemplary embodiment, not shown, the memory module and the calculation module form a common component of the analysis unit according to the present invention and are separated from one another at the software level only, and they are configured as a shared processor.

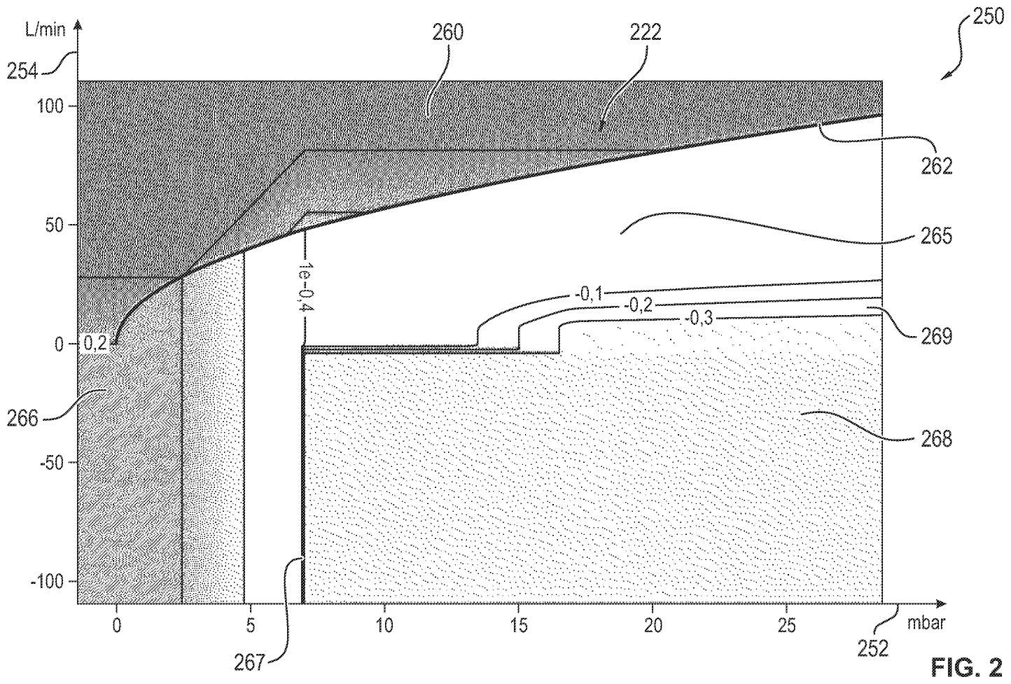

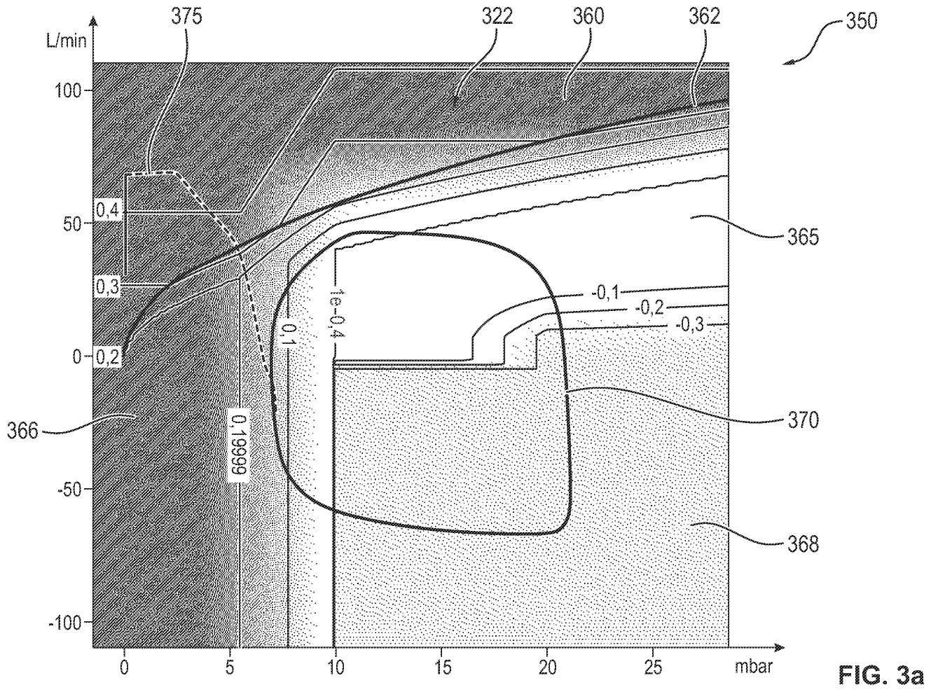

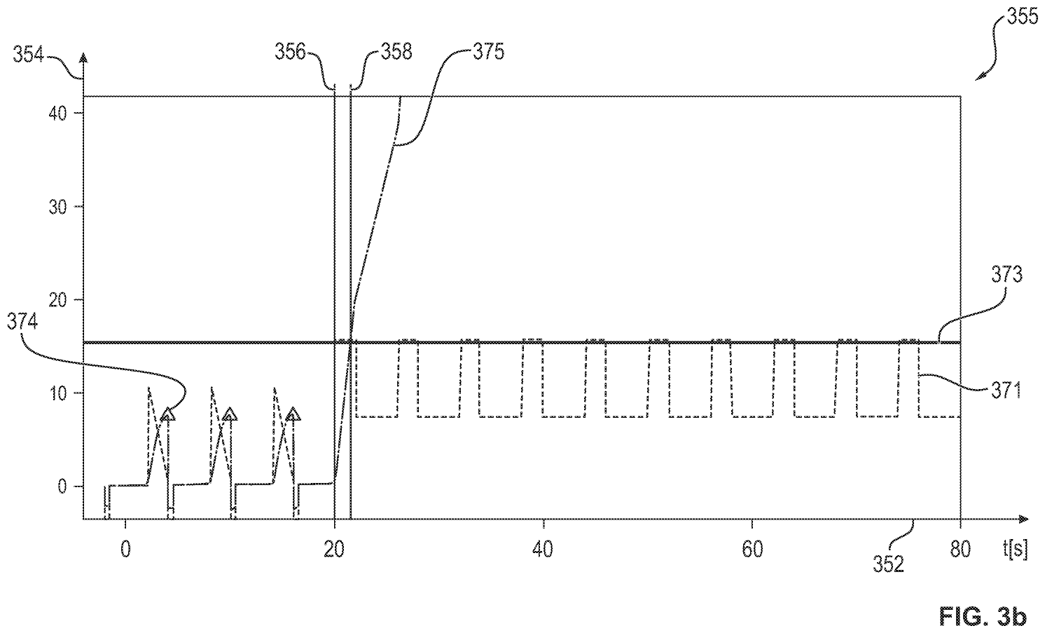

The calculation module 130 is configured to receive the gas flow data 112, the gas pressure data 114 and the current disconnection value function 122 and to calculate a current disconnection value D(P, F) 132 on the basis of the gas flow data 112 and the gas pressure data 114 via the current disconnection value function 122. Examples of such a disconnection value function 122 are explained within the framework of FIGS. 2 and 3. The calculation module 130 is further configured in this case to determine a disconnection number DN 134 from a chronological sequence of correspondingly calculated current disconnection values D(P, F) 132. This can be done, in this case, for example, by summation of the disconnection values D 132 from a predefined past time interval. The subscript m in FIG. 1 describes the number of disconnection values, which were calculated in the predefined past time interval. A fixed number of previously determined disconnection values are preferably used to determine the disconnection number. In one exemplary embodiment, not shown, only the time interval is set and the number of disconnection values used to determine the disconnection number may vary. The past time interval preferably equals at least 5 sec, especially at least 10 sec, and especially preferably at least 30 sec. As a result, the disconnection number 134 takes into account a plurality of breaths of the patient in order to determine whether a disconnection is present or not. As a result, the presence of a disconnection is detected especially reliably and false alarms are avoided. As an alternative or in addition, a low-pass filtering of past disconnection values may take place, so that very rarely occurring disconnection values are filtered out in order then to indicate only a disconnection via a corresponding output when a plurality of disconnection values indicate a corresponding disconnection. In another alternative or additional exemplary embodiment, the taking into account of the disconnection number is carried out by a so-called leaky integrator, in which the past disconnection values are weighted differently, namely, such that disconnection values located farther away in the past receive a lower weighting factor than more recent disconnection values. These differently weighted disconnection values are, in turn, summed up to form the disconnection number.

Finally, the calculation module 130 is configured to indicate the presence of a disconnection of the pneumatic connection via a corresponding output 140 if the disconnection number DN 134 reaches a predefined threshold value or is above this predefined threshold value over a predefined time period.

The predefined time period preferably equals less than 1 min, especially less than 30 sec, and especially preferably less than 10 sec.

The calculation of the disconnection value D 132 is preferably carried out essentially in real time. A time shorter than 10 sec, especially shorter than 5 sec and especially preferably shorter than 2 sec elapses for this between the receipt of the gas flow data 112 and of the gas pressure data 114 and the calculation of the disconnection value 132.

The analysis unit 100 according to the present invention is used within a ventilator, not shown. The individual modules are arranged in this case in a common housing of the ventilator and are configured by a shared processor, not shown, and they are separated from one another at least at the software level. In one exemplary embodiment, not shown, the modules are arranged at spaced locations from one another.

The calculation of the disconnection value 132 via the disconnection value function 122 may take place separated in space from the calculation of the disconnection number 134. Hence, the calculation module 130 may be configured as a plurality of submodules, which carry out a respective calculation step for the analysis unit according to the present invention. The calculation steps of the calculation module 130 are preferably carried out by a shared processor.

The data acquisition module 110 is configured to determine the gas flow signal 113, which can be read for the calculation module 130, from the number of received signals, which have the gas flow data 112 and the gas pressure data 114, and the gas pressure signal 115, which can be read for the calculation module 130. This may take place in one exemplary embodiment, not shown, via a receipt of these data without further processing steps. In another exemplary embodiment not shown, the received signals must be read out at discrete time steps in order to convert a continuous data stream of gas pressure data and gas flow rate into a sequence of discrete measured values.

The gas flow data and the gas pressure data indicate according to the present invention a state in the area of a transition between patient and ventilator. This transition is provided typically by a hose system. The received gas flow data and gas pressure data may correspond at least partially to measured values, which were recorded by a corresponding sensor mechanism in the area of this transition. As an alternative or in addition, these measured values may also be recorded in another area of the hose system and taken into account such that they at least indicate the actual state in the area of the transition between the patient and the ventilator. Such a taking into account may take place, for example, before or after the receipt by the data acquisition module according to the present invention. The data acquisition module may thus be configured to take into account the received gas flow data and the received gas pressure data such that they represent the respective state at the transition to the patient. The received data indicate this state according to the present invention by making possible such a taking into account. The disconnection value function is preferably selected such that it is adapted to the sensor mechanism providing the gas flow data and to the gas pressure data and a further taking into account of the received data is not necessary.

FIG. 2 shows a diagram 250 of a first exemplary embodiment of a disconnection value function 222 for the analysis unit according to the first aspect of the present invention.

The diagram 250 shows along its X axis 252 the current gas pressure in the unit mbar. A range from −1 mbar to 27 mbar along the X axis 252 is shown here.

Furthermore, diagram 250 shows along its Y axis 254 the current gas flow in the unit L/min. A range from −100 L/min to 100 L/min is shown here along the Y axis 254.

It can be seen on the basis of the contour lines shown which disconnection value is associated corresponding to the disconnection value function 222 shown with a state within the diagram 250 shown. A state corresponds here to a value pair of gas flow and gas pressure.

The upper area 260 of this diagram is separated via a quadratic function 262 between gas pressure and gas flow from a central area 265, which corresponds to a disconnection value of 0. The upper area 260 corresponds, by contrast, to a high disconnection value, which leads to overshooting of the threshold value for the disconnection number in a correspondingly occurring state already after a short time and correspondingly outputs the detection of a disconnection. High values for the gas flow suggest that no or at least only a low resistance is offered by the respiratory system of the patient. Such high values for the gas flow therefore indicate that a hose is not probably connected in this case any longer or that there is a disconnection of comparable gas-carrying equipment and they should lead in a short time to an alarm based on a probable disconnection. This is even more true for a low gas pressure because a low gas pressure and a high gas flow are typical signs of an open end of a gas connection. It is preferably assumed that a defined minimum impedance must be present for a certain pressure difference within a gas line. This minimum impedance may depend on different boundary conditions of the current treatment, e.g., the status of the patient, a type of hose or the like. This pressure difference-dependent minimum impedance is preferably given based on a quadratic function, as a result of which the quadratic function 262 between the gas pressure and the gas flow can be seen within the diagram 250.

The central area 265 is the inner area, in which a majority of a breathing cycle of a patient should take place with a ventilator connected. The disconnection value function is not preferably positive in this desired state in order not to reach over time the threshold value for the detection of a disconnection, without the usual range for the breathing cycle having been left. A disconnection value 0 is preferably associated with this central area 265 in order to make possible an especially rapid determination of the disconnection number by some summands already being zero at the time of the calculation.

Furthermore, a left area 266 of the diagram 250 can be seen, in which the disconnection value increases, so that a lasting state within this area leads after a certain time to a disconnection number that is above the predefined threshold value and therefore leads to the output of the suspected disconnection. This is due in the exemplary embodiment shown to the fact that a long-lasting gas pressure below a predefined gas threshold value, in this case especially above a predefined value for the positive end-expiratory pressure (PEEP) 267, indicates that a disconnection must have occurred because the PEEP value is typically the lowest value for the provided pressure in the course of a breathing cycle of the patient. Should, for example, a complete breath of the patient take place at a gas pressure below the PEEP, this is a comparatively reliable indicator that a disconnection has taken place. A short-term reduction of the gas pressure may, however, also indicate other causes, for example, coughing of the patient or the like, and it should not immediately lead to an output indicating the disconnection. Therefore, even though the disconnection value is positive in this area, it is not as high as in the upper area 260.

The lower area 268 leads to a negative disconnection value, and hence to a reduction of the disconnection number in the exemplary embodiment shown, in which the disconnection number is determined by summing up past disconnection values. This is due to the fact that a negative gas flow for a gas pressure that is not too low indicates that breathing takes place against the breathing gas provided, which indicates, in turn, that the patient is not yet connected to the ventilator. This does not apply any longer to excessively low gas pressures, because, for example, other causes for a negative gas flow are also possible at a gas pressure close to zero.

The lower area 268 becomes slightly wider for high gas pressures, which leads to the widening 269. This is due to the fact that a certain pressure difference can be expected between inhalation and exhalation, so that a high gas pressure with a correspondingly negative disconnection value shall compensate a possibly following low gas pressure with a possibly positive disconnection value, so that great pressure differences during the breathing do not lead slowly to an increase in the disconnection number. Moreover, the widening 269 is characterized by high gas pressures and low gas flows. In case of a disconnection, the gas pressure would have to collapse at low gas flows, so that the widening 269 represents an area with certain connection.

The curve shown by the diagram 250 illustrates the functional relationship used, which is described by the disconnection value function between the gas flow, the gas pressure and the disconnection value to be assigned.

In one exemplary embodiment, not shown, the disconnection value function is formed by a combination of the upper area and the left area from the diagram 250. In another exemplary embodiment, not shown, the disconnection value function is formed by a combination of the upper area and the lower area from the diagram 250. In another exemplary embodiment, not shown, the disconnection value function is formed by a combination of the left area and the lower area from the diagram 250. A disconnection value different from zero is assumed in another exemplary embodiment, not shown, by the disconnection value function in the central area in which a connection between the patient and the ventilator is assumed. A negative value is preferably assumed in the central area, so that a short-term, greatly deviating measured value, for example, based on a measurement error, does not lead to a rapid rise of the disconnection number. Such a short-term, greatly deviating measured value can also be removed from the determination of the disconnection number by filtering in the area of the data acquisition module, for example, by a low-pass filter, and/or by filtering the calculated disconnection value, for example, by a low-pass filter. According to the present invention, a single value for the disconnection value, which indicates a disconnection, shall not lead to the outputting of the corresponding output. The disconnection number is determined therefore according to the present invention from a plurality of disconnection values.

The disconnection value function does not preferably associate with the current state of gas flow and gas pressure a disconnection value that is above the predefined threshold value.

The predefined threshold value depends on the selected disconnection value function. The predefined threshold value is preferably determined within the framework of a calibration of the analysis unit according to the present invention prior to a use of this analysis unit during the ventilation of a patient.

FIG. 3a shows a diagram 350 of a second exemplary embodiment of a disconnection value function 322 for the analysis unit according to the first aspect of the present invention. FIG. 3b shows a time curve 355 of the disconnection number, as it has been formed for a breathing cycle 370 shown in FIG. 3a with the corresponding disconnection value function by a corresponding summation of the disconnection values. A so-called leaky integrator, in which the old integrator value is limited before the addition of the current disconnection value such that it is not lower than -1, is used in the exemplary embodiment shown.

The disconnection value function 322 used in the diagram 350 corresponds approximately to the disconnection value function 222 shown in FIG. 2. For the likewise mentioned reasons, the disconnection value function 322 thus has the upper area 360, the central area 365, the left area 366 as well as the lower area 368. The quadratic function 362 is shown for a better comparability of the diagrams 250 and 350 from FIGS. 2 and 3a.

The differences from the disconnection value function 222 are due to the fact that the shown adapted association with disconnection values was generated based on past gas flow data and on past gas pressure data and/or based on provided input data. An adapted disconnection value function was formed hereby, which replaces the preceding disconnection value function 222 in the exemplary embodiment shown. The structure of such an analysis unit is shown, for example, in FIG. 4. In the exemplary embodiment shown, the disconnection value function 322 is an adapted disconnection value function, which was formed from the disconnection value function 22 based on the characteristic properties of the ventilation of a concrete patient. For example, the central area 365 has become smaller here, because it was detected by the analysis unit that the patient is breathing typically in a concrete area within this diagram and changes from this typical breathing already suggest the probability of a disconnection between patient and ventilator. The typical breathing cycle 370 is likewise shown in the exemplary embodiment shown. The typical breathing cycle 370 passes through both an area of positive disconnection values and an area of negative disconnection values, so that the disconnection number remains essentially zero over a breathing cycle.

Such an adaptation of the disconnection value function, especially such an automated adaptation, makes possible an especially reliable and patient-dependent detection of a disconnection between patient and ventilator.

In addition, an exemplary disconnection curve 375 of a disconnection is shown by broken lines, and it deviates from the typical breathing cycle 370 starting from the time of disconnection, so that the state in the diagram 350 is shifted towards low gas pressures and high gas flows and it remains there.

FIG. 3b shows how the disconnection becomes noticeable in the time curve 355 of the disconnection number.

The time curve shows the time on the X axis 352 in sec and the disconnection number on the Y axis 354.

The broken line 371 shows the curve of the disconnection value in this diagram 375 for the case in which a disconnection occurs at the disconnection time 356. The deviation from the typical breathing cycle 370 leads in this case to a shift of the periodic curve from the area before the disconnection time 356 towards higher disconnection values. The curve of the disconnection number arising from the disconnection values is shown as a dash-dotted line 375. This curve always has a maximum, which is below a predefined threshold value 373 and is shown as a triangle in FIG. 3b, before this disconnection time 356. The values for the disconnection value rise after the disconnection time 356 and the disconnection number correspondingly reaches the predefined threshold value 373 at the detection time 358, at which the disconnection is detected according to the present invention between the patient and the ventilator. In the exemplary embodiment shown, the disconnection number is a scaled sum of disconnection values, which were determined within a past time interval.

In one exemplary embodiment, not shown, a corresponding detection of the disconnection takes place only when the disconnection number was above the predefined threshold value over a predefined time period. This predefined time period is preferably a continuous time period. In an alternative or additional exemplary embodiment, the predefined time period is a duration that is formed by individual time segments, in which the threshold value is exceeded, preferably within a chronologically past analysis time range, which is to be taken into consideration.

FIG. 4 shows a schematic view of a second exemplary embodiment of the analysis unit 400 according to the first aspect of the present invention.

The analysis unit 400 differs from the analysis unit 100 shown in FIG. 1 in that it has an input module 480, which can receive a user input 482 via an input interface 484, in this case especially via a keyboard, via a data carrier interface and/or via a touch display and can output corresponding input data to the additional modules of the analysis unit, in this case especially to the memory module 420 and to the calculation module 430.

The user input 482 may pertain especially to a manual analysis of an earlier output concerning the presence of the disconnection and/or to a manual setting of the predefined threshold value for the disconnection number and output a corresponding calculation signal 486 to the calculation module 430. For example, a new predefined threshold value is thus set in this case, which is taken into consideration by the calculation module 430 for the future decision on whether a disconnection was detected or not.

Furthermore, the user input 482 may indicate a current ventilation mode of the ventilator 490, a current patient category of the patient, e.g., his age group, ventilation equipment being used, for example, a hose type, and/or a current ventilation parameter, for example, the PEEP value. Such a user input 482 leads to a corresponding memory signal 488 to the memory module 420 in order to determine from a group of stored disconnection value functions 425 the disconnection value function 122 to be used currently and to output this to the calculation module 430.

Furthermore, the analysis unit 400 differs from the analysis unit 100 in that it has a modeling module 495, which is configured to adapt the disconnection value function depending on past gas pressure data and/or on provided input data and to make these available to the memory module 430. The input data provided may be, for example, data that were received via the user input 482. The adaptation of the disconnection value function is preferably carried out in an automated manner. The modeling module 495 is connected in this case to the memory module 430 in order to receive the disconnection value function 122 currently being used and to determine the adapted disconnection value function on the basis of the disconnection value function 122 currently being used as well as on the basis of the data received currently for the gas flow and for the gas pressure, which are transmitted via the combined signal 418 to the calculation module 430 and to the modeling module 495. In an alternative or additional exemplary embodiment, the modeling module is connected directly to the calculation module in order to output the adapted disconnection value function directly to the calculation module.

In one exemplary embodiment, not shown, the modeling module is configured to determine the adapted disconnection value function via a neuronal network. The data received and the disconnection values calculated last are used in this case as input variables for the neuronal network in order to provide in an automated manner a change of the disconnection value function such that the adapted disconnection value function can detect the disconnection of the connection between the patient and the ventilator even more reliably. A patient-specific disconnection value function, which takes into consideration the typical breathing cycle of the patient being currently ventilated, is especially advantageously determined here.

Furthermore, the modeling module 495 is configured to access a minimal disconnection value function and/or a maximal disconnection value function if a predefined function characteristic of the disconnection value function adapted by the modeling module exceeds a predefined characteristic threshold value. For example, a narrow width of the middle area of the disconnection value function concerning the gas flow leads in this case to an application of the minimal disconnection value function in order to ensure a middle area, in which the disconnection value function is zero. In an alternative or additional exemplary embodiment, a large gradient of the disconnection value function and hence an excessively rapid rise or drop of the disconnection number leads in a taking into account of different disconnection values to the application of the minimal disconnection value function or of the maximum disconnection value function. These two limits for the adaptation of the disconnection value function ensure that the analysis unit according to the present invention meets clinical requirements imposed on the safety of the detection of a disconnection despite an automated adaptation of the disconnection value function.

Finally, FIG. 4 also shows that the analysis unit 400 is arranged within the ventilator 490, wherein the housing 492 is represented by broken lines. In addition, a sensor device 494 is shown here, which outputs the gas flow data 112 and the gas pressure data 114 to the data acquisition module 410. The further processing by the data acquisition module 410 and by the calculation module 430 takes place analogously to the corresponding modules of the analysis unit 100.

Moreover, an output module 497 is configured to receive the output 140 and to provide an acoustic output via a corresponding output device 498, in this case via a speaker, following detection of a disconnection. The output module 497 is a part of the analysis unit 400 in the exemplary embodiment shown. In one exemplary embodiment, not shown, the analysis unit is configured independently from an external output module and the output module is not a component of the analysis unit.

The input module 480, the sensor device 494 and the output module 497 are incorporated each in the housing 492 in the exemplary embodiment shown and they thus allow an access by a user from the outside. The sensor device 494 is preferably arranged in the area of a hose system, not shown, which is connected to the ventilator 490.

In one exemplary embodiment, not shown, the determination of the disconnection value function to be used is carried out via a measurement maneuver of the ventilator to determine a dead space, a resistance and/or a compliance of the hose system and/or of a patient interface. A current disconnection value function, which is to be used, is associated with the variable thus determined and is outputted for use to the calculation module.

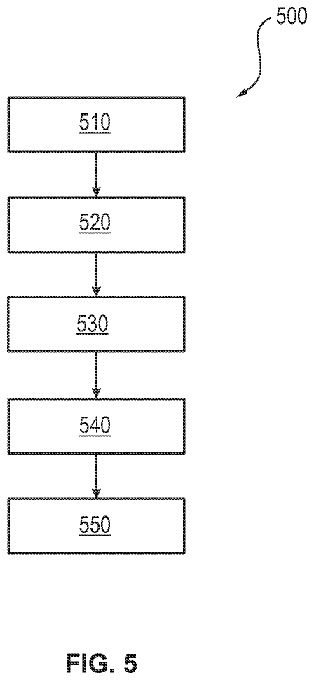

FIG. 5 shows a flow chart of an exemplary embodiment of a process 500 according to a third aspect of the present invention.

The process 500 according to the present invention is configured for the detection of a disconnection of a pneumatic connection between a ventilator and a patient to be ventilated by the ventilator. It has the steps explained below.

A first step 510 comprises a receipt of gas flow data and gas pressure data, the gas flow data indicating a gas flow present at the transition to the patient through the ventilator and the gas pressure data indicating a gas pressure present at the transition to the patient through the ventilator.

A next step 520 comprises a provision of a current disconnection value function, which describes an association between the gas flow present at the transition and the gas pressure present at the transition to a disconnection value to be associated (assigned).

A next step 530 comprises a calculation of a current disconnection value based on the gas flow data and on the gas pressure data via the current disconnection value function.

A further step 540 comprises a determination of a disconnection number from a chronological sequence of correspondingly calculated, current disconnection values.

A final step 550 comprises an indication of the presence of a disconnection of the pneumatic connection if the disconnection number reaches a predefined threshold value or it is above this predefined threshold value over a predefined time period. The steps of this process are thus carried out.

The provision of the disconnection value function 520 can be achieved by a calculation module accessing a stored value. The steps of this process are thus carried out compulsorily in the order indicated. After receiving the data in step 510, the disconnection value function is accessed within the framework of step 520 in order to determine the disconnection value in step 530. The taking into account of a chronological sequence of disconnection values is carried out again compulsorily before the disconnection number is compared in step 550 with a threshold value and after reaching the threshold value or after exceeding or undershooting the threshold value for a predefined time period and the output for indicating the currently probably existing disconnection is triggered.

Step 550 is carried out only rarely, namely, possibly only after the development of a disconnection between patient and ventilator. The further steps 510 through 540 of the process are preferably carried out essentially in real time. Thus, there is preferably a time period shorter than 10 sec, especially shorter than 5 sec and especially preferably shorter than 2 sec between the receipt of the gas flow data and of the gas pressure data and the determination of the current disconnection number. The time elapsing between the receipt of the gas flow data and the gas pressure data, on the one hand, and the determination of the disconnection value, on the other hand, is preferably shorter than 2 sec, especially shorter than 1 sec, and especially preferably shorter than 0.1 sec.

The disconnection number preferably also uses in this case the disconnection values, which were already used at the time of the determination of the previous disconnection number. After an initial determination of sufficient disconnection values to determine a disconnection number, the determination of a corresponding disconnection number is preferably carried out after each, after every second, after every third or after every fifth calculation of a disconnection value.

The first step 510 of the process according to the present invention may also be carried out while the last steps of the process according to the present invention, which were carried out before, have not yet been carried out. For example, the receipt of current gas flow data and gas pressure data may also continue, for example, during the determination of the disconnection number.

While specific embodiments of the invention have been shown and described in detail to illustrate the application of the principles of the invention, it will be understood that the invention may be embodied otherwise without departing from such principles.

### LIST OF REFERENCE NUMBERS

100, 400 Analysis unit
110, 410 Data acquisition module
112 Gas flow data
113 Gas flow signal
114 Gas pressure data
115 Gas pressure signal
120, 420 Memory module
122, 222, 322 Current disconnection value function
130, 430 Calculation module
132 Current disconnection value
134 Disconnection number
140 Output
250, 350 Diagram
252, 352 X axis
254, 354 Y axis
260, 360 Upper area
262 Quadratic function
265, 365 Middle area
266, 366 Left area
267 PEEP value
268, 368 Lower area
269 Widening
355 Time curve
356 Disconnection time
358 Detection time
370 Breathing cycle
371 Curve of the disconnection value in broken lines
373 Predefined threshold value
374 Maximum of the disconnection number
375 Example of disconnection curve
418 Combined signal
480 Input module
482 User input
484 Input interface
486 Calculation signal
488 Memory signal
490 Ventilator
492 Housing
494 Sensor device
495 Modeling module
497 Output module
498 Output device
500 Process
510, 520, 530, 540, Process steps
550

What is claimed is:

1. A ventilator analysis unit for detecting a disconnection of a pneumatic connection between a ventilator and a patient to be ventilated by the ventilator, the ventilator analysis unit comprising:
a data acquisition module configured to receive gas flow data indicating a gas flow present at a transition to the patient through the ventilator and to receive gas pressure data indicating a gas pressure present at the transition to the patient through the ventilator;
a memory module configured to provide a current disconnection value function, which describes an association between the gas flow present at the transition and the gas pressure present at the transition to a disconnection value to be assigned; and
a calculation module configured:
to receive the gas flow data, the gas pressure data and the current disconnection value function and to calculate a current disconnection value based on the gas flow data and the gas pressure data via the current disconnection value function;
to determine a disconnection number from a chronological sequence of correspondingly calculated current disconnection values; and
to indicate a presence of a disconnection of the pneumatic connection via a corresponding output if the disconnection number reaches or exceeds a predefined threshold value over a predefined time period.

2. A ventilator analysis unit in accordance with claim 1, wherein:
the data acquisition module is configured to receive the gas flow data and gas pressure data essentially in real time; and
the calculation module is configured to calculate the current disconnection value essentially in real time.

3. A ventilator analysis unit in accordance with claim 2, wherein the calculation module is configured to calculate the respective current disconnection values via a plurality of breaths of the patient and to determine the disconnection number based on the plurality of breaths.

4. A ventilator analysis unit in accordance with claim 1, wherein the disconnection value function depends on at least one of a current ventilation mode of the ventilator, a current patient category of the patient, ventilation equipment being used and a current ventilation parameter.

5. A ventilator analysis unit in accordance with claim 1, wherein the disconnection value function is based at least partially on a currently maximally expectable gas flow, which depends on the currently present gas pressure.

6. A ventilator analysis unit in accordance with claim 1, wherein the disconnection value function is based at least partially on a minimally expectable gas pressure.

7. A ventilator analysis unit in accordance with claim 1, further comprising an input module configured to receive a user input via an input interface and to provide a number of input data indicated by the user input.

8. A ventilator analysis unit in accordance with claim 7, wherein the user input pertains to a manual analysis of an output concerning at least one of a presence of a disconnection and a manual setting of the predefined threshold value for the disconnection number.

9. A ventilator analysis unit in accordance with claim 1, further comprising a modeling module configured to adapt the disconnection value function, depending on at least one of past gas flow data, past gas pressure data and provided input data indicated by a user input and to make the adapted disconnection value function available to the memory module, wherein the memory module is further configured to replace a current disconnection value function by the adapted disconnection value function.

10. A ventilator analysis unit in accordance with claim 9, wherein the adaptation of the disconnection value function by the modeling module is based on a processing of the disconnection value function by a neuronal network.

11. A ventilator analysis unit in accordance with claim 9, wherein:
a minimal disconnection value function is stored for the modeling module;
the modeling module is configured to provide as the adapted disconnection value function the minimal disconnection value function if a predefined function characteristic of the adapted disconnection value function, adapted by the modeling module, exceeds a predefined characteristic threshold value.

12. A ventilator comprising a ventilator analysis unit for detecting a disconnection of a pneumatic connection between a ventilator and a patient to be ventilated by the ventilator, the ventilator analysis unit comprising:
a data acquisition module configured to receive gas flow data indicating a gas flow present at a transition to the patient through the ventilator and to receive gas pressure data indicating a gas pressure present at the transition to the patient through the ventilator;
a memory module configured to provide a current disconnection value function, which describes an association between the gas flow present at the transition and the gas pressure present at the transition to a disconnection value to be assigned; and
a calculation module configured:
to receive the gas flow data, the gas pressure data and the current disconnection value function and to calculate a current disconnection value based on the gas flow data and the gas pressure data via the current disconnection value function;
to determine a disconnection number from a chronological sequence of correspondingly calculated current disconnection values; and
to indicate a presence of a disconnection of the pneumatic connection via a corresponding output if the disconnection number reaches or exceeds a predefined threshold value over a predefined time period.

13. A ventilator in accordance with claim 12, further comprising a sensor device configured to detect and to provide the gas flow data and the gas pressure data.

14. A ventilator in accordance with claim 13, wherein:
the data acquisition module is configured to receive the gas flow data and gas pressure data essentially in real time; and
the calculation module is configured to calculate the current disconnection value essentially in real time.

15. A ventilator in accordance with claim 13, wherein the disconnection value function depends on at least one of a current ventilation mode of the ventilator, a current patient category of the patient, ventilation equipment being used and a current ventilation parameter.

16. A ventilator in accordance with claim 13, wherein the disconnection value function is based at least partially on a currently maximally expectable gas flow, which depends on the currently present gas pressure or is based at least partially on a minimally expectable gas pressure.

17. A ventilator in accordance with claim 13, wherein the ventilator analysis unit further comprises an input module configured to receive a user input via an input interface and to provide a number of input data indicated by the user input.

18. A ventilator in accordance with claim 13, wherein the ventilator analysis unit further comprises a modeling module configured to adapt the disconnection value function, depending on at least one of past gas flow data, past gas pressure data and provided input data indicated by a user input and to make the adapted disconnection value function available to the memory module, wherein the memory module is further configured to replace a current disconnection value function by the adapted disconnection value function.

19. A process for detecting a disconnection of a pneumatic connection between a ventilator and a patient to be ventilated by the ventilator, the process comprising the steps of:
receiving gas flow data and gas pressure data, wherein the gas flow data indicates a gas flow present at a transition to the patient through the ventilator and wherein the gas pressure data indicates a gas pressure present at the transition to the patient through the ventilator;
providing a current disconnection value function, which describes an association between the gas flow present at the transition and the gas pressure present at the transition to a disconnection value to be assigned;
calculating a current disconnection value based on the gas flow data and on the gas pressure data via the current disconnection value function;
determining a disconnection number from a chronological sequence of correspondingly calculated, current disconnection values; and
indicating a presence of a disconnection of the pneumatic connection if the disconnection number reaches or exceeds a predefined threshold value over a predefined time period.

20. A process according to claim 19, wherein a program code is provided for carrying out at least some of the process steps when the program code is executed on a computer, on a processor or on a programmable hardware component.
